(19) United States
(12) Patent Application Publication
(10) Pub. No.: US 2006/0149364 A1
(43) Pub. Date: Jul. 6, 2006

(54) Low profile vascular graft

(76) Inventors: Steven Walak, Natick, MA (US); John Sherry, Needham, MA (US)

Correspondence Address: HOFFMANN & BARON, LLP, 6900 JERICHO TURNPIKE, SYOSSET, NY, 11791, US

(21) Appl. No.: 11/026,642

(22) Filed: Dec. 31, 2004

(51) Int. Cl. A61F 2/06

(52) U.S. Cl. 623/1.44

(57) ABSTRACT

The low profile vascular graft of the present invention includes a tube structure having outer and inner surfaces, and a support structure having a chamber structure secured to the outer or inner surface. The support structure includes a core structure contained within the chamber structure. The core structure is transformable from a conformance condition to a reinforcement condition. When the core structure is in the conformance condition, it provides insubstantial resistance to deformation of the tube structure. When the core structure is in the reinforcement condition, it provides substantial resistance to deformation of the tube structure.

### FIELD OF THE INVENTION

The present invention relates to a low profile vascular graft and, more specifically, to a reinforced vascular graft having a profile which may be lowered for insertion into and translation through the body of a patient.

### BACKGROUND OF THE INVENTION

Implantable vascular grafts are used in medical applications for the treatment of diseased or damaged blood vessels, such as arteries and veins. Such treatment may be necessitated by conditions in the arteries and veins, such as a stenosis, thrombosis, occlusion or aneurysm. A vascular graft may be used to repair, replace, or otherwise correct a diseased or damaged blood vessel.

A vascular graft may be a tubular prosthesis for replacement or repair of a damaged or diseased blood vessel. A vascular graft may be used in the vascular system, urogenital tract and bile duct, as well as in a variety of other applications in the body. A vascular graft may be reinforced to open and support various lumens in the body. Such a vascular graft may be used for the treatment of stenosis, strictures and aneurysms in blood vessels, such as arteries and veins. Such treatments include implanting the vascular graft within the blood vessel to open and/or reinforce collapsing or partially occluded sections of the vessel.

The opening and reinforcing of sections of lumens in the body, such as blood vessels, is frequently accomplished by using vascular grafts which themselves have additional support structures, such as stents. Such support structures resist deformation of the open internal passage through the vascular graft. This provides the desired opening and reinforcement of the body lumens through which such vascular grafts extend.

However, the resistance to deformation provided by the support structure may inhibit insertion of the vascular graft into the body since the opening in the body may have a shape which differs from the cross-sectional shape of the vascular graft which is maintained by the support structure. Accordingly, undesired deformation of the opening in the body may be required to insert the vascular graft having such additional support.

Additionally, the resistance to deformation provided by the support structure may reduce the flexibility of the vascular graft. This may result in forcible contact between the vascular graft and interior sections of the body lumen during translation of the vascular graft through the body lumen since the internal contour and direction of the body lumen typically varies. Such variation frequently results in inclined or even direct orthogonal contact between the vascular graft and internal surface of the body lumen. Such contact may result in deformation of the body lumen if the vascular graft is relatively inflexible.

### SUMMARY OF THE INVENTION

The low profile vascular graft of the present invention includes a tube structure having outer and inner surfaces, and a support structure having a chamber structure secured to the outer or inner surface. The support structure includes a core structure contained within the chamber structure. The core structure is transformable from a conformance condition to a reinforcement condition. When the core structure is in the conformance condition, it provides insubstantial resistance to deformation of the tube structure. When the core structure is in the reinforcement condition, it provides substantial resistance to deformation of the tube structure.

The insubstantial resistance to deformation provided by the core structure in the conformance condition enables the profile of the vascular graft to be lowered to conform to the shape of the opening in the patient's body through which the graft is inserted. Such insertion may be facilitated by the profile reduction by avoiding deformation of the opening in the patient's body which may otherwise be necessary to accommodate the cross-sectional shape of an inflexible vascular graft.

The insubstantial resistance to deformation provided by the core structure in the conformance condition also increases the longitudinal flexibility of the vascular graft. This facilitates translation of the graft through the body lumen since the vascular graft, upon encountering a changed contour or direction of the body lumen during translation therethrough, is able to flexibly deflect thereby reducing the magnitude of any deformation forces which could be imparted to the body lumen by such contact therewith by the graft.

The resistance to deformation provided by the core structure in the reinforcement condition provides an opening force to facilitate the reduction or removal of any obstruction or blockage in the section of the body lumen through which the vascular graft is inserted. Also, the resistance to deformation provided by the core structure supports the body lumen through which the vascular graft extends to facilitate the maintenance of the lumen in an open condition.

The transformability of the core structure enables the vascular graft to be inserted into and translated through the body lumen with the core structure in the conformance condition. This provides the low profile and flexibility to the vascular graft which facilitates the insertion and translation.

When the vascular graft has reached the desired location in the body lumen, the transformability allows the core structure to be transformed to the reinforcement condition. This provides the resistance to deformation of the vascular graft which facilitates the reduction or removal of any obstruction or blockage in the body lumen and maintenance thereof in the open condition.

These and other features of the invention will be more fully understood from the following description of specific embodiments of the invention taken together with the accompanying drawings.

### BRIEF DESCRIPTION OF THE DRAWINGS

In the drawings:

FIG. 1 is a perspective view of a low profile vascular graft of the present invention, the graft being shown as having a tube structure and a support structure on the outer surface thereof;

FIG. 2 is a cross-sectional view of the vascular graft of FIG. 1 in the plane indicated by line 2-2 of FIG. 1;

FIG. 3 is a cross-sectional view of the vascular graft of FIG. 1 in the plane indicated by line 3-3 of FIG. 1;

FIG. 4 is a perspective view of an alternative second embodiment of the vascular graft of FIG. 1, the graft being shown as having core elements within the support structure;

FIG. 5 is a cross-sectional view of the vascular graft of FIG. 4 in the plane indicated by line 5-5 of FIG. 4;

FIG. 6 is a cross-sectional view of the vascular graft of FIG. 4 in the plane indicated by line 6-6 of FIG. 1;

FIG. 7 is an enlarged perspective view of a portion of the support structure of FIG. 4, the core elements being shown in the conformance condition;

FIG. 8 is an enlarged perspective view of the portion of the support structure of FIG. 7, the core elements being shown in the reinforcement condition;

FIG. 9 is an enlarged perspective view of a portion of an alternative embodiment of the support structure of FIG. 7, the core elements being shown in the conformance condition;

FIG. 10 is an enlarged perspective view of the portion of the support structure of FIG. 9, the core elements being shown in the reinforcement condition;

FIG. 11 is a perspective view of an alternative third embodiment of the vascular graft of FIG. 1, the graft being shown as having a support structure which is helical;

FIG. 12 is a cross-sectional view of the vascular graft of FIG. 11 in the plane indicated by line 12-12 of FIG. 11.

FIG. 13 is a cross-sectional view of the vascular graft of FIG. 11 in the plane indicated by line 13-13 of FIG. 11;

FIG. 14 is a schematic view of an alternative embodiment of the support structure of FIG. 1, the core structure being shown in the conformance condition;

FIG. 15 is a schematic view of the support structure of FIG. 14, the core structure being shown in the reinforcement condition in which the core structure does not contact the chamber structure;

FIG. 16 is a perspective view of an alternate embodiment of the support structure of FIG. 1, the support structure being shown assembled before being secured to the tube structure;

FIG. 17 is a block diagram showing a method for making the support structures, including the support structures of FIGS. 1 to 19;

FIG. 18 a perspective view of an alternative fourth embodiment of the vascular graft of FIG. 1, the support structure being shown as located between outer and inner tube structures;

FIG. 19 is an elevation view of the distal end of the vascular graft of FIG. 18;

FIG. 19a is a perspective view of an alternative fifth embodiment of the vascular graft of FIG. 1, the support structure being shown as located between outer and inner tube structures;

FIG. 19b is an elevation view of the distal end of the vascular graft of FIG. 19a;

FIG. 19c is a perspective view of an alternative sixth embodiment of the vascular graft of FIG. 1, the support structure being shown as located on the outer and inner surfaces of the tube structure;

FIG. 19d is an elevation view of the distal end of the vascular graft of FIG. 19c;

FIG. 19e is a perspective view of an alternative seventh embodiment of the vascular graft of FIG. 1, the support structure being shown as located on the outer and inner surfaces of the tube structure;

FIG. 19f is a schematic view of a portion of the distal end of the vascular graft of FIG. 19e, the support structure on the inner surface of the graft being shown in the conformance condition;

FIG. 19g is a schematic view of the distal end of the vascular graft of FIG. 19e, the support structure on the inner surface of the graft being shown in the reinforcement condition;

FIG. 20 is a block diagram showing a method for making the vascular graft of FIG. 18; and

FIG. 21 is a block diagram showing an alternative second embodiment of the method of FIG. 20; and

FIG. 22 is a block diagram showing an alternative third embodiment of the method of FIG. 20.

Corresponding reference characters indicate corresponding parts throughout the several views of the drawings.

### DETAILED DESCRIPTION OF THE INVENTION

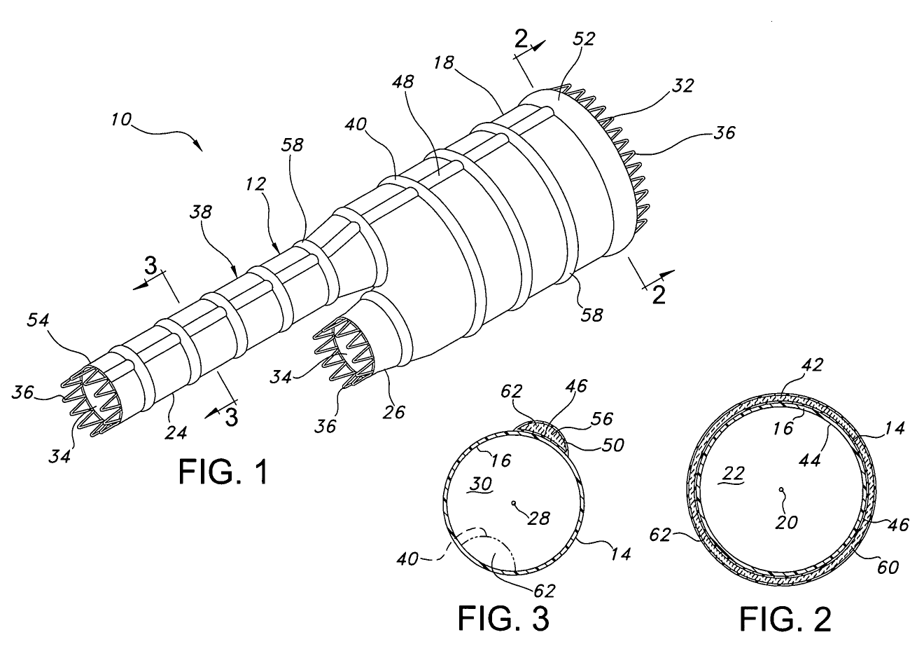

Referring to the drawings and more particularly to FIGS. 1 and 2, a low profile vascular graft 10 is shown as having a tube structure 12 which may be formed of polytetrafluoroethylene (PTFE) material. The tube structure 12 has outer and inner surfaces 14, 16, and a trunk portion 18 which has a longitudinal central axis 20 and an interior region 22. The tube structure 12 also includes a pair of leg portions 24, 26, each of which has respective longitudinal central axis 28 and interior region 30. The leg portions 24, 26 extend from one of the ends of the trunk portion 18 such that the interior regions 30 of the leg portions communicate with the interior region 22 of the trunk portion.

The ends of the tube structure 12 which are opposite from the connection of the trunk portion 18 to the leg portions 24, 26 define proximal and distal ends 32, 34 of the tube structure 12. For example, the end of the trunk portion 18 which is opposite to the leg portions 24, 26 may constitute the proximal end 32 of the tube structure 12. The ends of the leg portions 24, 26 which are opposite to the trunk portion 18 may constitute the distal ends 34 of the tube structure 12.

The vascular graft 10 includes stents 36 connected at the proximal and distal ends 32, 34 of the tube structure 12. The stent 36 connected to the proximal end 32 is connect to the trunk portion 18. The stents 36 connected to the distal ends 34 are connected to both of the leg portions 24, 26.

The vascular graft 10 has a support structure 38 including a chamber structure 40 secured to the outer surfaces 14 of the trunk portion 18 and leg portions 24. Additionally, the chamber structure 40 may be secured to the inner surface 16 of the tube structure 12, as shown in FIG. 3.

The chamber structure 40 has outer and inner surfaces 42, 44. The inner surface 44 bounds an interior cavity 46 within the chamber structure 40. The volume of the interior cavity 46 defines the internal volume of the chamber structure 40. Expansion of the internal volume of the chamber structure 40 is limited.

The chamber structure 40 may include a longitudinal chamber 48 which has a longitudinal central axis 50 which extends in the same direction as the central axes 20, 28 of the trunk portion 18 and leg portion 24. The longitudinal chamber 48 has a proximal end 52 which is adjacent to the proximal end 32 of the tube structure 12. The longitudinal chamber 48 has a distal end 54 which is adjacent to the distal end 34 of the tube structure 12. The longitudinal chamber 48 may extend continuously between the proximal and distal ends 52, 54 and thereby extends over substantially the entire length of the trunk portion 18 and leg portion 24. The longitudinal chamber 48 has an interior cavity 56.

The chamber structure 40 includes circular chambers 58 around the trunk portion 18 and both of the leg portions 24, 26. The circular chambers 58 are spaced longitudinally and may intersect the longitudinal chamber 48. Each of the circular chambers 58 has an interior cavity 60. The cavities 56, 60 may be connected with one another at the junctions between the longitudinal chamber 48 and circular chambers 58 to provide for communication between the cavities.

The support structure 38 includes a core structure 62 contained within the chamber structure 40. In a preferred embodiment, the core structure 62 is a one-piece core element which extends through the respective cavities 56, 60 of the longitudinal and circular chambers 48, 58 which communicate with one another.

The core structure 62 is a super-expanding material such as highly elastic polymers, shape memory polymers, nitinol, super absorbent polymers, and super absorbent hydrogels. The material of the core structure 62 can further be formed into foams, felts, and open spheres to provide the highest level of expansion possible. The core structure 62 has an external volume which is no greater than the internal volume of the chamber structure 40 when the core structure has not been expanded. When the core structure 62 is unexpanded, the external volume thereof is substantially less than the internal volume of the chamber structure 40. This provides a clearance between the core structure 62 and inner surface 44 of the chamber structure 40 resulting in flexibility thereof. This enables the core structure 62 to conform to a variety of contours such as encountered by the tube structure 12 within the body of a patient, and establishes the core structure, when not expanded, as being in a conformance condition.

The core structure 62 may be expanded sufficiently for engagement thereof with the inner surface 44 of the chamber structure 40. Such expansion of the core structure 62 is sufficient for the engagement thereof with the chamber structure 40 to be with sufficient force to provide substantial resistance to deformation of the tube structure. This resistance to deformation provides reinforcement to the tube structure 12 and establishes the core structure, when expanded, as being in a reinforcement condition.

Accordingly, the core structure 62 is transformable from a conformance condition to a reinforcement condition. When the core structure 62 is in the conformance condition, such as if the core structure is a super absorbent material and such material is either dry or slightly moist, the core structure 62 provides insubstantial resistance to deformation of the tube structure 12. When such a core structure 62 is in the reinforcement condition, such as by absorbing a sufficient quantity of liquid, the core structure 62 provides substantial resistance to deformation of the tube structure 12. This resistance to deformation may be provided by the chamber structure 40 being secured to either the outer or inner surfaces 42, 44.

The expansion the core structure 62 may be triggered according to various mechanisms. This transforms the core structure 62 from the conformance condition to the reinforcement condition. For example, the material of the core structure 62 may be selected such that absorption thereof by a sufficient amount of liquid, such as blood or other body fluids, causes the super-expansion of the core structure. Provision of liquid to the core structure 62, to cause such super-expansion, may be by forming the chamber structure 40 of a permeable material. When such a chamber structure 40 is inserted into the body of a patient, blood or other body fluids contact the outer surface 42, permeate through the chamber structure and inner surface 44 and enter the interior cavities 56, 60. This exposes the core structure 62 to the liquid and, after sufficient absorption thereof by the core structure, results in the core structure transforming from the conformance condition to the reinforcement condition.

Other mechanisms for triggering the expansion of the core structure 62 for the transformation thereof from the conformance condition to the reinforcement condition include the release of mechanical constraint applied to the core structure, actuation of shape change materials, and water absorption by the core structure. Additional mechanisms include heating, light activation, and a change in pH of the core structure.

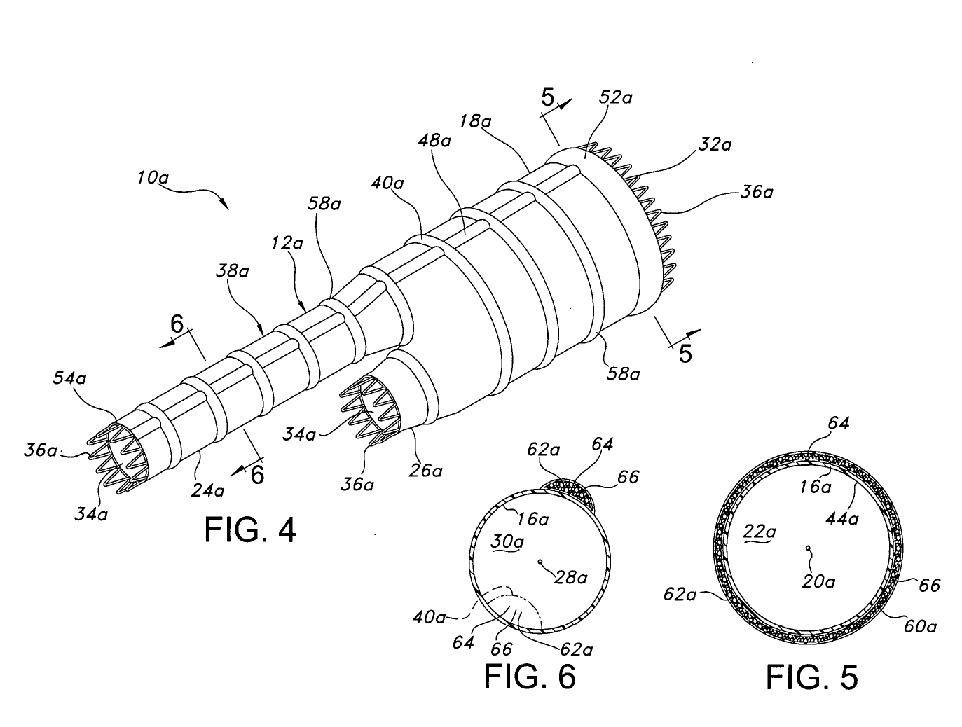

An alternative embodiment of the vascular graft 10a is shown in FIGS. 4 to 6. The vascular graft 10a includes a tube structure 12a which has outer and inner surfaces 14a, 16a, and a trunk portion 18a. In these and additional respects, the vascular graft 10a corresponds to the vascular graft 10. Accordingly, parts illustrated in FIGS. 4 to 6 which correspond to parts illustrated in FIGS. 1 to 3 have, in FIGS. 4 to 6, the same reference numeral as in FIGS. 1 to 3 , with the addition of the suffix “a”.

The core structure 62a includes a group of core elements 64 contained within the longitudinal and circular chambers 48a, 58a. Such core elements 64 are formed of super-expanding or shape memory materials which may be expanded from a conformance condition to a reinforcement condition. The core elements 64 form a cluster 66 which has an external volume which is no greater than the internal volumes of the longitudinal and circular chambers 48a, 58a when the core elements are in the conformance condition. Preferably, the external volume of the cluster 66 is substantially less than the internal volumes of the longitudinal and circular chambers 48a, 58a when the core elements are in the conformance condition. When the core elements 64 are transformed from the conformance to reinforcement conditions thereof, the cluster 66 sufficiently expands to engage the inner surface 44a of the chamber structure 40a with sufficient force to provide substantial resistance to deformation of the tube structure 12a.

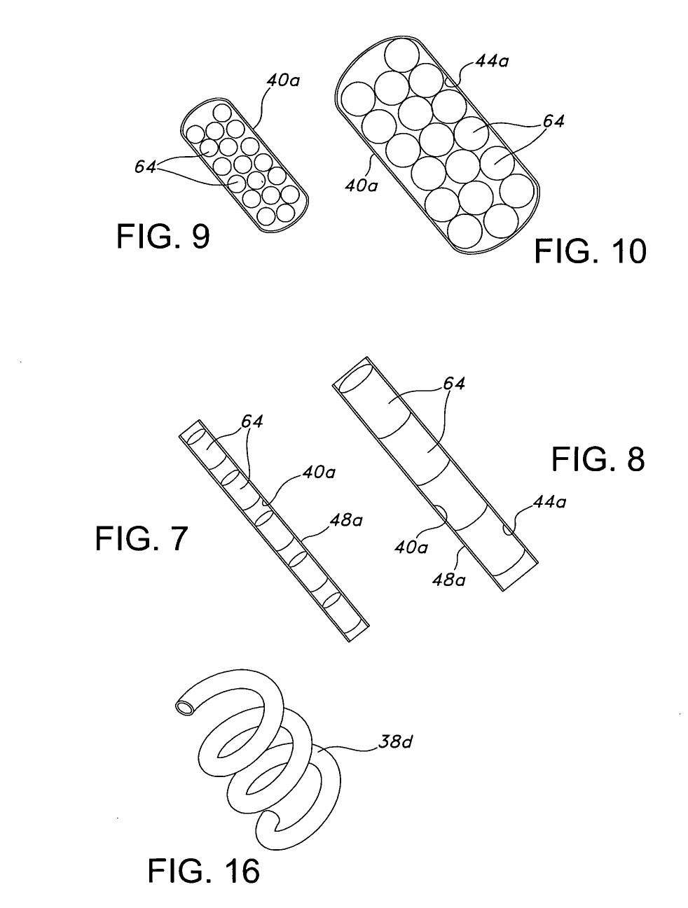

FIGS. 7 and 8 show the longitudinal chamber 48a and the core elements 64 contained therein. FIG. 7 depicts the core elements 64 in the conformance condition, before expansion thereof. FIG. 8 illustrates the core elements 64 of FIG. 7 in the reinforcement condition after expansion thereof. Expansion of the core elements 64 may result in corresponding expansion of the chamber structure 40a, as shown in FIG. 8. FIGS. 9 and 10 illustrate a further embodiment of the core elements 64 in the conformance and reinforcement conditions, respectively.

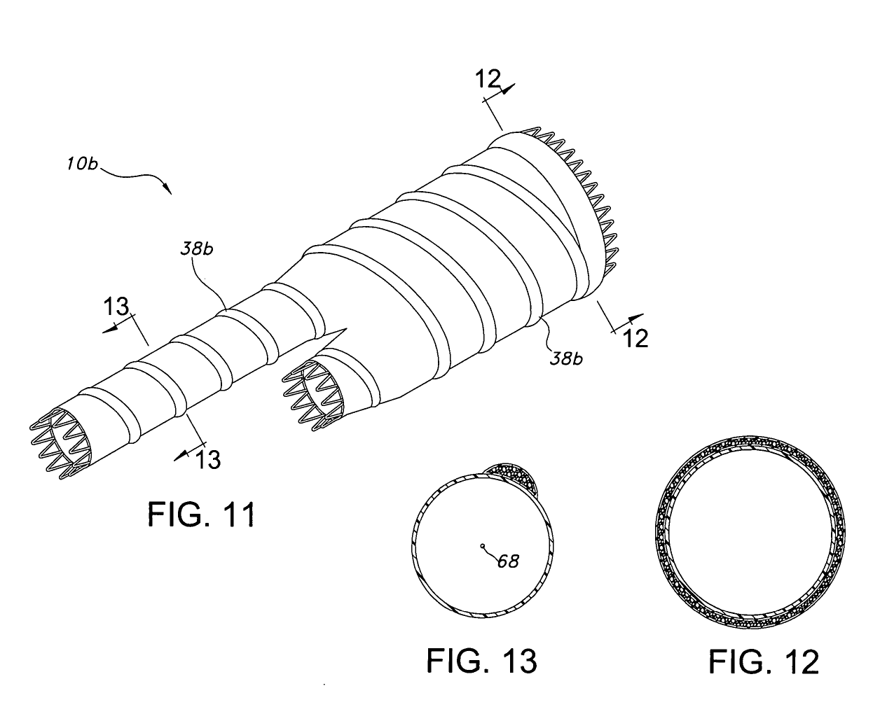

An alternative embodiment of the vascular graft 10b is shown in FIGS. 11 to 13. The vascular graft 10b includes a tube structure 12b which has outer and inner surfaces 14b, 16b, and a trunk portion 18b. In these and additional respects, the vascular graft 10b corresponds to the vascular graft 10. Accordingly, parts illustrated in FIGS. 11 to 13 which correspond to parts illustrated in FIGS. 1 to 3 have, in FIGS. 11 to 13, the same reference numeral as in FIGS. 1 to 3, with the addition of the suffix “b”. The support structure 38b is helical and has longitudinal central axes 68 which substantially coincide with the longitudinal central axis 20b of the trunk portion 18b and the longitudinal central axes 28b of the leg portions 24b, 26b of the tube structure 12b.

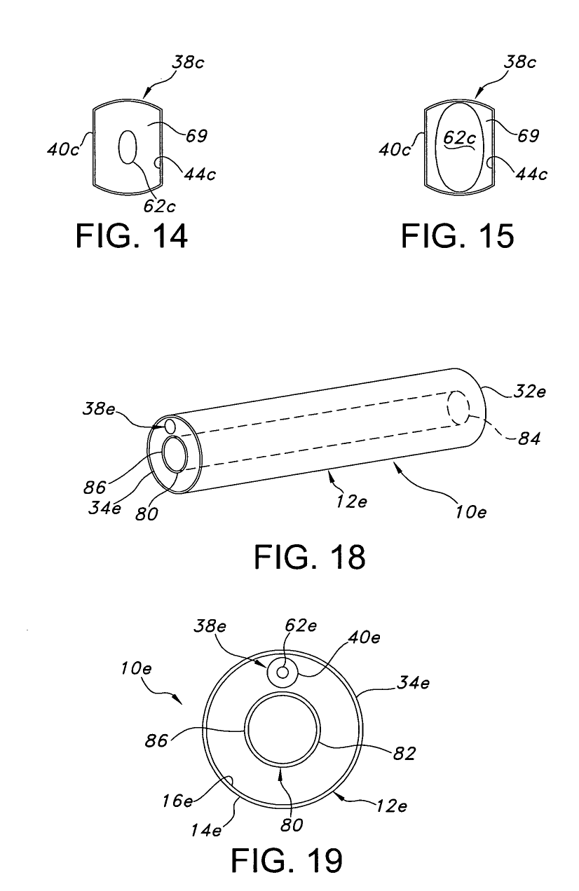

An alternative embodiment of the support structure 38c is shown in FIGS. 14 and 15. The support structure 38c includes a chamber structure 40c and core structure 62c. In these and other respects, the support structure 38c corresponds to the support structure 38. Accordingly, parts illustrated in FIGS. 14 and 15 which correspond to parts illustrated in FIGS. 1 to 3 have, in FIGS. 14 and 15, the same reference numeral as in FIGS. 1 to 3, with the addition of the suffix “c”. The transformation of the core structure 62c from the conformance to reinforcement conditions increases the pressure 69 within the chamber structure 40c to provide substantial resistance to deformation of the tube structure 12c. Such an increase in pressure may not require direct contact of the core structure 62c with the inner surface 44c of the chamber structure 40c. This increase in pressure may be provided by the chamber structure 40c, and core structure 62c being sufficiently impermeable to gas and liquid, and any expansion of the chamber structure being sufficiently limited. As a result, when the core structure 62c begins to expand to the reinforcement condition, an increased pressure is transmitted to the inner surface 44c of the chamber structure 40c. This increase in pressure provides substantial resistance to deformation of the tube structure 12c.

In further alternative embodiments of the vascular graft, such as the graft 10, the chamber structure, such as structure 40, may include a plurality of longitudinal chambers, such as chamber 48. Also, the chamber structure may have multiple interior cavities, such as cavity 46. Additionally, the longitudinal and circular chambers may have multiple cavities, such as cavities 56, 60. Moreover, communication between one or more of the cavities may be obstructed. Also, the chamber and core structures, such as structures 40, 62, may be impermeable, such as to liquid and gas.

A support structure 38d may be pre-fabricated and assembled before attachment thereof to the tube structure 12. The support structure 38d, shown in FIG. 16, includes a chamber structure 40d and core structure 62d. In these and other respects, the support structure 38d corresponds to the support structure 38. Accordingly, parts illustrated in FIG. 16 which correspond to parts illustrated in FIGS. 1 to 3 have, in FIG. 16, the same reference numeral as in FIGS. 1 to 3, with the addition of the suffix “d”. The support structure 38d may include a chamber structure 40d which includes a thin walled elastomer tube having a diameter of approximately 0.062 inches. Such a chamber structure 40d would be filled with a core structure 62d constituted by super-expanding particulate. The support structure 38d, including the chamber structure 40d and core structure 62d, could be pre-fabricated in relatively long lengths and stored until assembly to the tube structure 12. Such a support structure 38d could be helical, as shown in FIG. 16.

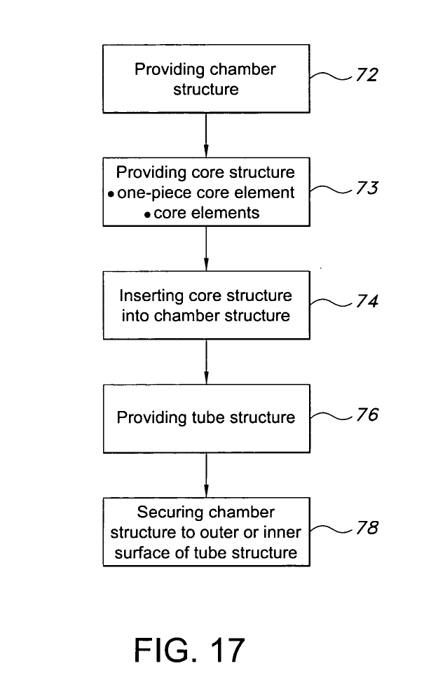

The pre-fabricated support structures, including the support structures 38a to 38e, may be made and secured to a tube structure 12 according to the method designated generally by the reference numeral 70 in FIG. 17. The method 70 includes providing 72 a chamber structure 40 and providing 73 a core structure 62. The core structure 62 is then inserted 74 into the chamber structure 40. A tube structure 12 having outer and inner surfaces 14, 16 is then provided 76 according to the method 70. The chamber structure 40 is then secured 78 to the outer or inner surface 14, 16 of the tube structure 12.

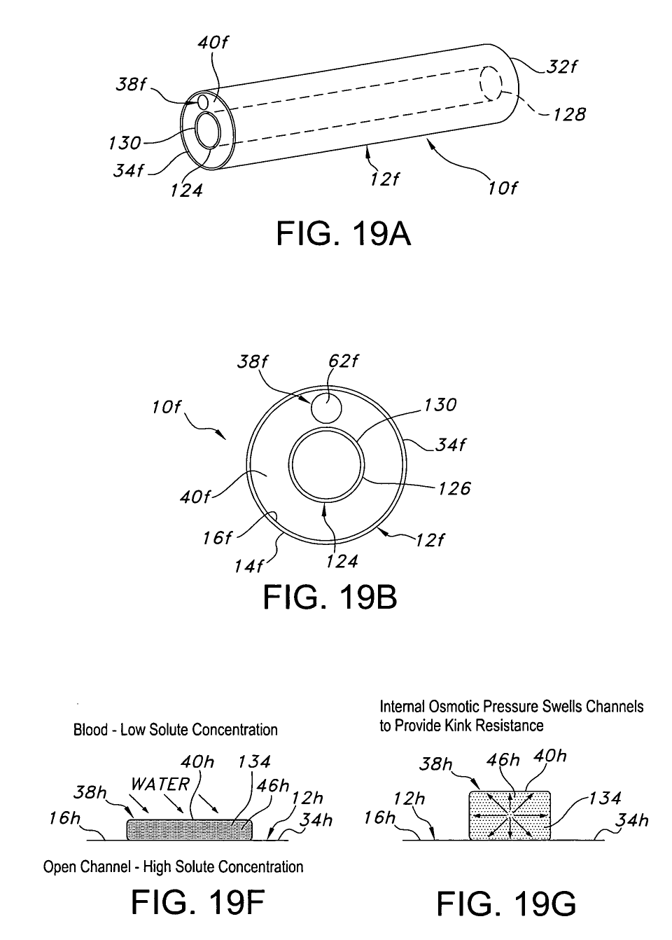

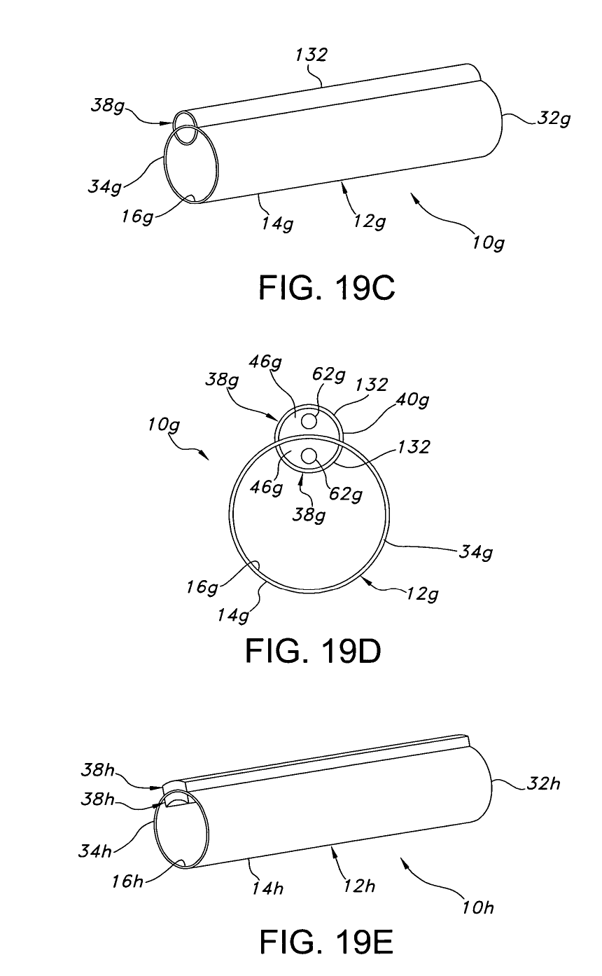

In an alternative embodiment shown in FIGS. 18 and 19, the vascular graft 10e may include an outer tube structure 12e which corresponds to the tube structure 12 in FIGS. 1 to 3. A support structure 38e which corresponds to the support structure 38 in FIGS. 1 to 3 is secured to the inner surface 16e of the outer tube structure 12e. In these and additional respects, the vascular graft 10e corresponds to the vascular graft 10. Accordingly, parts illustrated in FIGS. 18 and 19 which correspond to parts illustrated in FIGS. 1 to 3 have, in FIGS. 18 and 19, the same reference numeral as in FIGS. 1 to 3, with the addition of the suffix “e”.

The vascular graft 10e includes an inner tube structure 80 having an outer surface 82 and proximal and distal ends 84, 86. The inner tube structure 80 is within the outer tube structure 12e in coaxial relation therewith such that the proximal ends 32e, 84 of the outer and inner tube structures 12e, 80 longitudinally coincide relative to one another. The distal ends 34e, 86 of the outer and inner tube structures 12e, 80 longitudinally coincide relative to one another. The inner and outer surfaces 16e, 82 are bonded to one another to fix the longitudinal coincidence of the proximal ends 32e, 84 relative to one another and the longitudinal coincidence of the distal ends 34e, 86 relative to one another. Examples of the outer and inner tube structures 12e, 80 including materials and methods for assembly thereof are disclosed in U.S. Patent Application Publication No. US 2003/0204241, the entire disclosure of which is hereby incorporated by reference herein.

The support structure 38a, which includes a chamber structure 40a and core structure 62a therein, is secured to one or both of the inner and outer surfaces 16a, 82 such that the support structure is between the outer and inner tube structures 12a, 80. The core structure 62a is transformable from a conformance condition to a reinforcement condition. The core structure 62a provides substantial resistance to deformation of the outer and inner tube structures 12a, 80 when the core structure is in the reinforcement condition.

In an alternative embodiment shown in FIGS. 19a and 19b, the vascular graft 10f may include an outer tube structure 12f which corresponds to the tube structure 12 in FIGS. 1 to 3. A support structure 38f which corresponds to the support structure 38 in FIGS. 1 to 3 is located between the inner surface 16f of the outer tube structure 12f. In these and additional respects, the vascular graft 10f corresponds to the vascular graft 10. Accordingly, parts illustrated in FIGS. 19a and 19b which correspond to parts illustrated in FIGS. 1 to 3 have, in FIGS. 19a and 19b, the same reference numeral as in FIGS. 1 to 3, with the addition of the suffix “f”.

The vascular graft 10f includes an inner tube structure 124 having an outer surface 126 and proximal and distal ends 128, 130. Examples of the outer and inner tube structures 12f, 124 including materials and methods for assembly thereof are disclosed in U.S. Patent Application Publication No. US 2003/0204241. The inner tube structure 124 is within the outer tube structure 12f in coaxial relation therewith such that the proximal ends 32f, 128 of the outer and inner tube structures 12f, 124 longitudinally coincide relative to one another. The distal ends 34f, 130 of the outer and inner tube structures 12f, 124 longitudinally coincide relative to one another.

A radial clearance is provided between the outer and second tube structures 12f, 124 such that the radial clearance defines the chamber structure 40f. The outer and inner tube structures 12f, 124 are bonded to one another to maintain the chamber structure 40f and fix the longitudinal coincidence of the proximal ends 32f, 128 relative to one another and the longitudinal coincidence of the distal ends 34f, 130 relative to one another. The chamber structure 40f is sealed 122 to contain the core structure 62f therein. The core structure 62f may be a one-piece core element, or may include a plurality of core elements.

In an alternative embodiment shown in FIGS. 19c and 19d, the vascular graft 10g may include a tube structure 12g which corresponds to the tube structure 12 in FIGS. 1 to 3. Support structures 38g which correspond to the support structure 38 in FIGS. 1 to 3 are located on the outer and inner surfaces 14g, 16g of the tube structure 12g. In these and additional respects, the vascular graft 10g corresponds to the vascular graft 10. Accordingly, parts illustrated in FIGS. 19c and 19d which correspond to parts illustrated in FIGS. 1 to 3 have, in FIGS. 19c and 19d, the same reference numeral as in FIGS. 1 to 3, with the addition of the suffix “g”.

Each of the chamber structures 40g is formed by a layer 132 which is bonded to the outer or inner surfaces 14g such that the interior cavity 46g is defined by the inner surface of the layer and the portion of the outer surface 14g, 16g which is enclosed by the layer. The layer 132 may be formed of an elastic material in close or adjoining contact with the core structure 62g. Upon activation of the core structure 62g, such as by expansion thereof, the layer 132 will expand to a fixed transverse dimension, such as a fixed diameter. Increased internal pressure, such as the pressure within the chamber structure 40g, due to the elastic recoil of the layer 132 will provide structural support and resistance to deformation of the tube structure 12g.

In an alternative embodiment shown in FIGS. 19e and 19f, the vascular graft 10h may include a tube structure 12h which corresponds to the tube structure 12 in FIGS. 1 to 3. Support structures 38h which correspond to the support structure 38 in FIGS. 1 to 3 are located on the outer and inner surfaces 14h, 16h of the tube structure 12h. In these and additional respects, the vascular graft 10h corresponds to the vascular graft 10. Accordingly, parts illustrated in FIGS. 19e and 19f which correspond to parts illustrated in FIGS. 1 to 3 have, in FIGS. 19e and 19f, the same reference numeral as in FIGS. 1 to 3, with the addition of the suffix “h”.

The chamber structure 40h is provided by a semi-permeable membrane which contains a material 134 which, when the chamber structure is inserted into the body of a patient, will cause fluid flow through the semi-permeable membrane into the interior cavity 46h to provide substantial resistance to deformation of the tube structure 12h. Such resistance to deformation may result from an increase in the pressure within the chamber structure 40h. The material 134 may be a solute, the concentration of which within the chamber structure 40h, before contact of the chamber structure with blood, is higher than the solute concentration in blood.

The chamber structure 40h, immediately after insertion of the graft 10h into the body of a patient, is illustrated schematically in FIG. 19f. The semi-permeability of the membrane of the chamber structure 40h allows fluid, such as water, to flow through the membrane into the interior cavity 46h. Consequently, the chamber structure 40h expands, as shown in FIG. 19g. This provides structural support and resistance to deformation of the tube structure 12h.

The one or more semi-permeable membranes of the chamber structure 40h, which may be considered “expansion channels”, create osmotic pressure and swelling thereof for the structural support of devices that may include AAA stent-grafts. This results from fluid from the blood stream being drawn into the “expansion channel” by a chemical gradient. The chemical driving force may be created by establishing a solute concentration differential or surface activation across the membrane.

The osmotic pressure created across the semi-permeable membrane of the chamber structure 40h causes channel filling and structural integrity without additional physician intervention. Osmotic pressure developed across the semi-permeable membrane of the chamber structure 40h forms structurally rigid tubular members, such as the tube structure 12h in the body of the patient without physician intervention.

A fixation stent may attached to a covering with open channels. The “open channel” structure of the chamber structure 40h is formed by a semi-permeable membrane on the blood contacting side. In one embodiment, an albumin concentration gradient is established across the membrane and drives the flow of water from the blood plasma into the “open channels” of the chamber structure 40h. Osmotic pressure developed inside the “open channels” force the channels to swell and become rigid providing support for the body of the structure of the graft 10h, such as the tube structure 12h.

Osmotic pressure can be developed by preloading the semi-permeable channels of the chamber structure 40h with a higher concentration of solute that is present in the blood. In one embodiment, a membrane that allows the free flow of water but prevents the flow of albumin is used to create an “open channel” in the chamber structure 40h of the graft 10h. Concentrations of albumin greater than that present in the blood will cause water to flow from the blood into the “channel” of the chamber structure 40h. Osmotic pressure in the channel will provide structural support, such as to the tube structure 12h, without requiring separate injection of materials, such as polymers, into the chamber structure 40h, and the preparation of such material for such injection. Solute concentration gradients based on albumin, glucose, sucrose, Ca⁺ or K⁺ could be used with appropriate semi-permeable membranes.

Nanomax polyamide membranes produced by Millipore could be used for the chamber structure 40h with the larger solute molecules albumin, sucrose or glucose. These membranes prevent transport of larger molecules but allow the free flow of water.

The “channel support” structure of the chamber structure 40h could be formed in rings or could be more extensive. A fully supported double wall tube-like device may provide superior kink resistance to a channel structure. Alternative membranes and solute molecules are possible. Active transport membranes which “pump” water under thermal or electrical activation may be used to substantially eliminate the need for solute within the channel of the chamber structure 40h. The chamber structure 40h may include semi-permeable ePTFE membranes. A preferred embodiment of the chamber structure 40h would include semi-permeable ePTFE membranes provided such membranes are available in the proper pore size. The chamber structure 40h may include active transport membranes.

Possible uses of the chamber structure 40h include the support surgical grafts, and distal filters. Embolic spheres that expand under developed internal osmotic pressure would facilitate sealing.

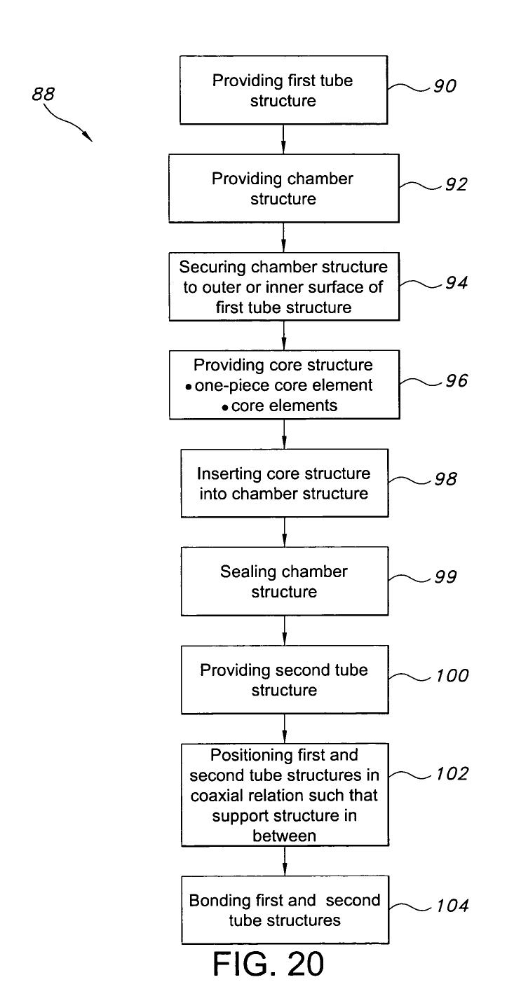

A low profile vascular graft 10 including outer and inner tube structures 12a, 80 may be made according to the method designated generally by the reference numeral 88 in FIG. 20. The method 88 includes providing 90 a first tube structure, such as the outer tube structure 12a, having outer and inner surfaces, such as the outer and inner surfaces 14a, 16a. The chamber structure of a support structure, such as the chamber structure 40a of the support structure 38a, is then provided 92. The chamber structure is next secured 94 to the outer or inner surface of the first tube structure. The method 88 then includes providing 96 a core structure of the support structure which is a one-piece core element, such as the core structure 62 of the support structure 38. Alternatively, the core structure may include a plurality of core elements, such as the core elements 64. Next, the core structure is inserted 98 into the chamber structure. Then, the chamber structure is sealed 99 to contain the core structure therein. A second tube structure, such as the inner tube structure 80, is then provided 100. The first tube structure is next positioned 102 in coaxial relation to the second tube structure, such as the inner tube structure 80, such that the support structure is between the first and second tube structures. Then, the first and second tube structures are bonded 103 to one another.

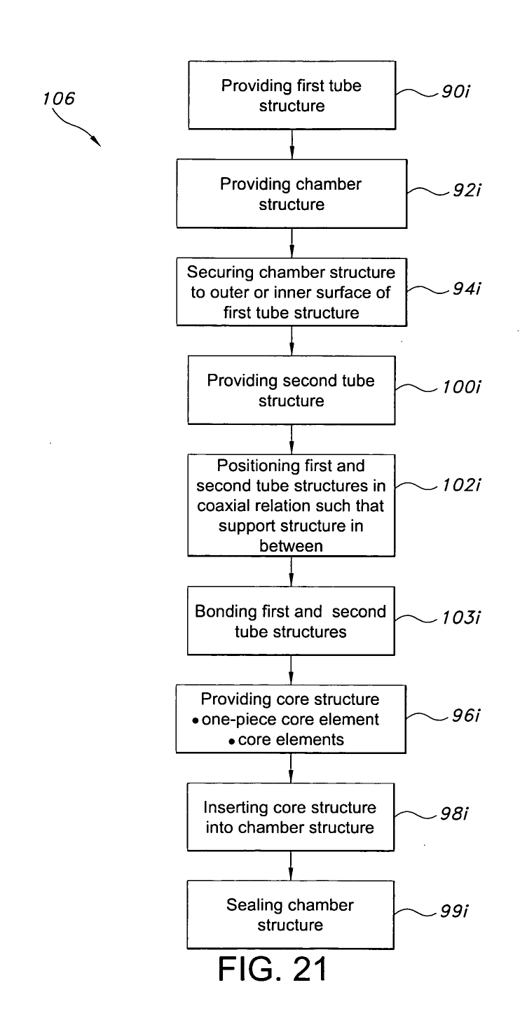

A low profile vascular graft 10 including outer and inner tube structures 12a, 80 may also be made according to the method designated generally by the reference numeral 106 in FIG. 21. The method 106 includes the step of providing 90f a first tube structure having outer and inner surfaces. In these and additional respects, the steps of the method 106 correspond to the method 88. Accordingly, the steps of the method 106 which correspond to steps of the method 88 have, in FIG. 21, the same reference numeral as in FIG. 20, with the addition of the suffix “i”. The method 106 provides for the bonding together of the first and second tube structures before the provision 96i of the core structure which includes a plurality of core elements, such as the core elements 64. Alternatively, the core structure may be a one-piece core element, such as the core structure 62. Following this, the core structure is inserted 98i into the chamber structure. Then, the chamber structure is sealed 99i to contain the core structure therein.

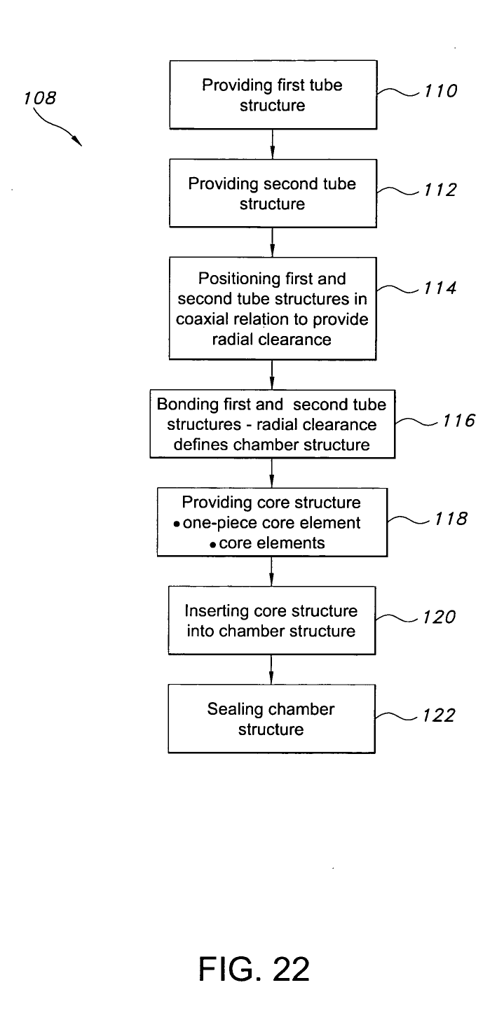

A low profile vascular graft 10f, as shown in FIGS. 19a and 19b, may be made according to the method designated generally by the reference numeral 108 in FIG. 22. The method 108 includes the steps of providing outer and inner tube structures. Following this, the inner tube structure is positioned 114 within and in coaxial relation to the outer tube structure to provide a radial clearance between the inner and outer tube structures. Next, the inner and outer tube structures are bonded together 116 such that the radial clearance defines a chamber structure. Then, a core structure is provided 118. The core structure may be a one-piece core element, such as the core structure 62a, or the core structure may include a plurality of core elements, such as the core elements 64. Following this, the core structure is inserted 120 into the chamber structure and the chamber structure is sealed 122 to contain the core structure therein.

The vascular graft 10 may be provided for insertion into the body of a patient with the core structure 62 in the conformance condition. This facilitates translation of the graft 10 through the lumen in the body of the patient since the core structure 62 provides insubstantial resistance to deformation of the tube structure 12. Deformation of the vascular graft 10 is normally required during such insertion because the body lumen through which the graft is typically inserted normally changes in both direction and cross-section. After the vascular graft 10 has reached its desired location, the core structure 62 is transformed from the conformance condition to the reinforcement condition. When in the reinforcement condition, the core structure 62 provides increased resistance to deformation of the tube structure 12.

The support structure 38 provides control over the timing of the transformation so that the core structure 62 remains in the conformance condition until the vascular graft 10 has reached its desired location. This typically requires a delay between the initial entry of the vascular graft 10, including the core structure 62, into the body lumen and the transformation. This may be provided, for example, for a core structure 62 which is so transformed by absorption thereof of fluids in the body, by the controlling the permeability of the chamber structure 40. More specifically, the permeability of the chamber structure 40 may be sufficiently limited to provide a delay between the immediate exposure of the outer surface 42 of the chamber structure 40 to the blood and the other body fluids, and the absorption thereof by the core structure 62 in a sufficient amount for the transformation thereof from the conformance condition to the reinforcement condition.

The entire disclosure of U.S. Pat. No. 6,395,019 is hereby incorporated by reference herein.

While the invention has been described by reference to certain preferred embodiments, it should be understood that numerous changes could be made within the spirit and scope of the inventive concept described. Accordingly, it is intended that the invention not be limited to the disclosed embodiments, but that it have the full scope permitted by the language of the following claims.

What is claimed is:

1. A low profile vascular graft comprising:
a tube structure having outer and inner surfaces, and a longitudinal axis; and
a support structure comprising a chamber structure secured to said outer or inner surface, said chamber structure having a longitudinal axis which extends in substantially the same direction as said longitudinal axis of said tube structure,
said support structure further comprising a core structure contained within said chamber structure wherein said core structure is transformable from a conformance condition to a reinforcement condition, said core structure providing insubstantial resistance to deformation of said tube structure when said core structure is in said conformance condition, said core structure providing substantial resistance to deformation of said tube structure when said core structure is in said reinforcement condition.

2. A low profile vascular graft according to claim 1, wherein said chamber structure has an internal volume the expansion of which is limited, said core structure comprising a super-expanding material which has an external volume that is no greater than said internal volume when said core structure is in said conformance condition, said transforming of said core structure to said reinforcement condition causing sufficient expansion of said core structure for engagement thereof with said chamber structure with sufficient force to provide said substantial resistance to deformation of said tube structure.

3. A low profile vascular graft according to claim 2, wherein said external volume of said core structure when in said conformance condition is substantially less than said internal volume of said chamber structure.

4. A low profile vascular graft comprising:
a tube structure having outer and inner surfaces; and
a support structure comprising a chamber structure secured to said outer or inner surface,
said support structure further comprising a core structure contained within said chamber structure wherein said core structure comprises a plurality of core elements, said core elements being transformable from a conformance condition to a reinforcement condition, said core elements providing insubstantial resistance to deformation of said tube structure when said core elements are in said conformance condition, said core elements providing substantial resistance to deformation of said tube structure when said core elements are in said reinforcement condition.

5. A low profile vascular graft according to claim 4, wherein said chamber structure has an internal volume the expansion of which is limited, said core elements comprising a super-expanding material and forming a cluster which has an external volume that is no greater than said internal volume when said core elements are in said conformance condition, said transforming of said core elements to said reinforcement condition causing sufficient expansion of said core elements for engagement of said cluster with said chamber structure with sufficient force to provide said substantial resistance to deformation of said tube structure.

6. A low profile vascular graft according to claim 5, wherein said external volume of said cluster when said core elements are in said conformance condition is substantially less than said internal volume of said chamber structure.

7. A low profile vascular graft comprising:
a tube structure having outer and inner surfaces; and
a support structure comprising a chamber structure secured to said outer or inner surface,
said support structure further comprising a core structure contained within said chamber structure wherein said core structure is substantially impermeable, said core structure being transformable from a conformance condition to a reinforcement condition, said core structure providing insubstantial resistance to deformation of said tube structure when said core structure is in said conformance condition, said core structure providing substantial resistance to deformation of said tube structure when said core structure is in said reinforcement condition.

8. A low profile vascular graft according to claim 7, wherein said chamber structure has an internal volume the expansion of which is limited, said core structure comprising a super-expanding material which has an external volume that is no greater than said internal volume when said core structure is in said conformance condition, said transforming of said core structure to said reinforcement condition causing sufficient expansion of said core structure for engagement thereof with said chamber structure with sufficient force to provide said substantial resistance to deformation of said tube structure.

9. A low profile vascular graft according to claim 8, wherein said external volume of said core structure when in said conformance condition is substantially less than said internal volume of said chamber structure.

10. A method for making a low profile vascular graft comprising:
providing a chamber structure of a support structure;
providing a core structure of the support structure;
inserting the core structure into the chamber structure;
providing a tube structure having outer and inner surfaces; and
securing the chamber structure to the outer or inner surface.

11. A method according to claim 10, wherein said step of providing a core structure comprises providing a one-piece core element.

12. A method according to claim 10, wherein said step of providing a core structure comprises providing a plurality of core elements.

13. A method according to claim 10, and further comprising the step of sealing the chamber structure to contain the core structure therein, said sealing step being after said inserting step and before said step of providing a tube structure.

14. A method for making a low profile vascular graft comprising:
providing a first tube structure having outer and inner surfaces;
providing a chamber structure of a support structure;
securing the chamber structure to the outer or inner surface;
providing a core structure of the support structure;
inserting the core structure into the chamber structure;
providing a second tube structure;
positioning the first tube structure in coaxial relation to the second tube structure such that the support structure is between the first and second tube structures; and
bonding the first and second tube structures to one another.

15. A method according to claim 14, wherein said step of providing a core structure comprises providing a one-piece core element.

16. A method according to claim 14, wherein said step of providing a core structure comprises providing a plurality of core elements.

17. A method according to claim 14, and further comprising the step of sealing the chamber structure to contain the core structure therein, said sealing step being after said inserting step and before said step of providing a second tube structure.

18. A method for making a low profile vascular graft comprising:
providing an outer tube structure;
providing an inner tube structure;
positioning the inner tube structure within and in coaxial relation to the outer tube structure to provide a radial clearance between the outer and inner tube structures;
bonding the inner and outer tube structures to one another such that the radial clearance defines a chamber structure;
providing a core structure of a support structure; and
inserting the core structure into the chamber structure.

19. A method according to claim 18, wherein said step of providing a core structure comprises providing a one-piece core element.

20. A method according to claim 18, wherein said step of providing a core structure comprises providing a plurality of core elements.

21. A method according to claim 18, and further comprising the step of sealing the chamber structure to contain the core structure therein, said sealing step being after said inserting step.

22. A low profile vascular graft comprising:
a tube structure having outer and inner surfaces; and
a support structure comprising a chamber structure secured to said outer or inner surface, said chamber structure having an inner surface which bounds an interior cavity within said chamber structure, said chamber structure comprising a semi-permeable membrane,
said support structure further comprising a material contained within said chamber structure which, when said chamber structure is inserted into the body of a patient, will cause fluid flow through said semi-permeable membrane into said interior cavity to provide substantial resistance to deformation of said tube structure.
